(19) United States
(12) Patent Application Publication
(10) Pub. No.: US 2006/0281782 A1
(43) Pub. Date: Dec. 14, 2006

(54) Method for the enantioselective preparation of sulphoxide derivatives

(76) Inventors: Avraham Cohen, Tel Aviv (IL); Suzy Charbit, Creteil (FR); Francois Schutze, Saint-Nom-La-Breteche (FR); Frederic Martinet, Paris (FR)

Correspondence Address: BUCHANAN, INGERSOLL & ROONEY PC, POST OFFICE BOX 1404, ALEXANDRIA, VA, 22313-1404, US

(21) Appl. No.: 10/551,037

(22) PCT Filed: Mar. 26, 2004
(86) PCT No.: PCT/FR04/00778

§ 371 Date: Jul. 26, 2006

(30) Foreign Application Priority Data
Mar. 28, 2003 (FR) 0303914
Dec. 15, 2003 (FR) 0314679

(51) Int. Cl. A61K 31/4745; A61K 31/4439; C07D 471/02; C07D 403/02

(52) U.S. Cl. 514/303; 514/338; 546/273.7; 546/118

(57) ABSTRACT

The invention relates to a method for the enantioselective preparation of substituted sulphoxide derivatives. The method comprises carrying out an enantioselective oxidation of a sulphide of general formula (I): A-CH2—S—B (I), where A=a variously-substituted pyridyl nucleus and B=a heterocyclic group with a benzimidazole or imidazopyrdyl nucleus, by means of an oxidising agent in the presence of a catalyst based on tungsten or vanadium and a chiral ligand, followed, where necessary, by salt formation with a base to give the sulphoxide: A-CH2—SO—B (Ia). The above is of application to the enantioselective preparation of compounds such as the enantiomers of tenatoprazole and other comparable sulphoxides.

The present invention concerns a method of enantioselective preparation of substituted derivatives of sulfoxides, and more particularly a method of enantioselective preparation of compounds such as the enantiomers of tenatoprazole and other similar compounds.

Several derivatives of sulfoxide, and particularly of pyridinyl-methyl-sulfinyl benzimidazoles are known to be useful in therapeutics, acting as drugs endowed with properties which inhibit proton pump, that is to say drugs that inhibit the secretion of gastric acid and are useful in the treatment of gastric and duodenal ulcers. The first known derivative of this series of proton pump inhibitors is omeprazole, or 5-methoxy-2-[[(4-methoxy-3,5-dimethyl-2-pyridinyl)methyl]sulfinyl]-1H-benzimidazole, described in Patent No. EP 005.129, which is endowed with properties which inhibit the secretion of gastric acid and is widely employed as an anti-ulcerant in human therapy. Other derivatives of benzimidazole are known by their generic names, for example rabeprazole, pantoprazole, lansoprazole, and all exhibit structural analogy and belong to the group of pyridinyl-methyl-sulfinyl-benzimi-dazoles.

Tenatoprazole, that is 5-methoxy-2-[[(4-methoxy-3,5-dimethyl-2-pyridyl)methyl]sulfinyl]imidazo[4,5-b]pyridine, is described in Patent No. EP 254.588. It is also part of the drugs considered as proton pump inhibitors, and it can also be used in the treatment of gastro-oesophageal reflux, digestive bleeding and dyspepsia.

All these compounds are sulfoxides presenting with asymmetry at the sulphur atom, and may therefore take the form of a racemic mixture of two enantiomers. It may be useful to separate them selectively in the form of any one enantiomer with R and S configurations, or (+) or (−) respectively, with specific properties that may be significantly different.

Several methods have been described in scientific literature to prepare either one of the enantiomers of these sulfoxides in a selective or predominant manner, especially omeprazole and its enantiomer of S configuration, esome-prazole, as well as its salts such as the sodium salt or magnesium salt.

Thus, Patent No. EP 652.872 describes the preparation method for the magnesium salt of the (−) enantiomer of omeprazole using its ester comprising a chiral acyloxymethyl group, separation of the diastereoisomers and solvolysis in an alkaline solution. U.S. Pat. No 5,776,765 describes a method which uses the stereoselective bioreduction of the racemic mixture of sulphide in the corresponding sulfoxide, using a microorganism containing a DMSO reductase, which enables to obtain a mixture that is considerably enriched with the (−) enantiomer, compared to the (+) enantiomer. U.S. Pat. No. 5,948,789 concerns the enantioselective preparation of sulfoxides, and particularly of the (−) enantiomer of omeprazole or of its sodium salts, via oxidation of the corresponding sulphide by a hydroperoxide in the presence of a titanium complex and of a chiral ligand. The method described in this patent makes it possible to obtain a mixture that is enriched with either one of the (−) and (+) enantiomers, according to the ligand used.

Works conducted so far by the applicant have showed that enantiomers of sulfoxide derivatives, and especially of tenatoprazole, can be obtained in an enantioselective manner under good purity and yield conditions, by enantioselective oxidation of the corresponding sulphide in the presence of a specific tungsten- or vanadium-based catalyst.

The present invention thus concerns an enantioselective preparation method for derivatives of sulfoxides presenting with asymmetry at the sulphur atom, producing either one of the enantiomers at a satisfactory level of yield and purity.

More particularly, this invention concerns a method of preparation which could produce in a noticeably enantio-selective manner the (−) and (+) enantiomers of tenatoprazole. The terms “in a noticeably enantioselective manner” used above means that the desired enantiomer is obtained in a selective manner or in predominant quantities compared to the other enantiomer.

According to the method of preparation of the invention, an enantioselective oxidation of a sulphide represented by the following general formula (I)
A-CH₂—S—B (I)
in which A is a pyridyl nucleus substituted in different ways and B a heterocylic residue comprising a benzimidazole or imidazo-pyridyl nucleus,
is carried out using an oxidizing agent in the presence of a tungsten- or vanadium-based catalyst and a chiral ligand, followed by salt formation by a base, if necessary.

In the general formula (I) hereabove, A represents preferably a pyridyl group or a pyridyl group bearing one or more substitutuents selected from the linear or branched alkyl groups of 1 to 6 carbon atoms, linear or branched alkoxy groups of 1 to 6 carbon atoms, methyl or ethyl groups substituted by one or several halogen atoms, amino, alkylamino or dialkylamino groups where the alkyl moiety, whether linear or branched, comprises of 1 to 5 carbon atoms; B represents a heterocycle selected from the benzimidazole or imidazo-[4,5-b]-pyridyl groups, substituted if necessary by one or several linear or branched alkyl groups of 1 to 6 carbon atoms, linear or branched alkoxy groups of 1 to 6 carbon atoms, and preferably substituted on one or several carbons by a methyl, ethyl, methoxy or trihalomethyl group.

In the general formula (I) here-above, A is preferably a 2-pyridyl group substituted by one or several methyl, ethyl, methoxy or trifluoromethyl groups, and more particularly a 4-methoxy-3,5-dimethyl-2-pyridyl group. B is preferably a 5-methoxy-1H-benzimidazolyle group or a B 5-methoxy-imidazo-[4,5-b]-pyridyl group.

The sulphide corresponding to the formula (I) here-above is a known product that can be prepared according to several methods described in literature, and for example, according to the methods described in Patents No. EP 254.588 and EP 103.553.

A sulfoxide is thus obtained which has the following formula
A-CH₂—SO—B (Ia)
wherein A and B have the definition given above.

The oxidant used in the method of the invention is preferably a peroxide, hydrogen peroxide for example, or a hydroperoxide, cumene or tertiobutyl hydroperoxide for example. According to an advantageous method of implementation, highly concentrated hydrogen peroxide, higher than 30% for example, or a hydrogen peroxide complexed with urea (UHP:urea hydrogen peroxide H₂NCONH₂.H₂O₂), herein after called <<UHP >>) is used.

The tungsten- or vanadium-based catalyst is an essential element of the method of the invention which allows for the reaction to take place and for the desired derivative to be obtained with a good yield. According to the invention, a catalyst such as a V oxo-vanadium complex, prepared from vanadium acetylacetonate VO(acac)₂, for example, or else a derivative of tungsten such as tungsten trioxide WO₃, is preferably used. Such catalysts are commercially available. A complex prepared from vanadium sulphate VOSO₄ can also be used.

The choice of the ligand constitutes another characteristic element of the invention since it allows for the reaction to be selectively directed towards the desired enantiomer.

According to the present invention, in the case of a vanadium-based catalyst, the ligand is preferably tridentate.

The ligand can be advantageously represented by the following general formula (II):
RO—CR₁R₂—CR₃R₄—NR₅R₆ (II)
where R is a hydrogen atom or a linear or branched alkyl group of 1 to 6 carbon atoms or an aryl or heteroaryl group;
R₁ to R₄, which can be the same or different, represent a linear or branched alkyl group of 1 to 6 carbon atoms, possibly comprising a heteroatom such as sulphur, nitrogen and oxygen and/or substituted by an amino group; an aryl group; an alkylaryl group; an alkoxycarbonyl group; a heteroaryl group or a heterocyle; or a heteroarylalkyl or a heterocyclalkyl group, with the proviso that R₁ should not be identical with R₂, and/or R₃ should not be identical with R₄, so that the ligand comprises one, or two asymmetry centers;
R₁ and R₂ together can represent a carbonyl group C═O;
R₁ and R₃, or R₂ and R₄ together, can form a carbon ring having 5 or 6 carbon atoms or a bicyclic system with 9 or 10 carbon atoms where one of the cycles can be aromatic;
Similarly, R₄ and R₅ can form a 5- or 6-membered heterocycle with the nitrogen atom;
R₅ and R₆, whether identical or different, represent a linear or branched alkyl group of 1 to 6 carbon atoms or a 5 or 6-membered carbon ring, or form a heterocycle with the nitrogen atom to which they are bound, or R₅ and R₆ represent, together with the nitrogen, a —N═CHAr double bond where Ar is a aryl residue, possibly substituted by 1 to 3 groups, and preferably bearing a hydroxyl group.

Preferably, Ar is a 2′-hydroxyphenyl group possibly substituted on the aryl group.

R₁ and R₃, or R₂ and R₄, represent preferably a hydrogen atom, whereas R₂ and R₄, or R₁ and R₃, respectively, are linear or branched alkyl groups of 1 to 6 carbon atoms, a aryl group or form together a carbon ring having 5 or 6 carbon atoms or a bicyclic system with 9 or 10 carbon atoms where one of the cycles can be aromatic.

According to the present invention:

an <<aryl group >> means preferably a mono- or poly-cyclic ring system having one or more aromatic rings including phenyl group, naphtyl group tetrahydronaphtyl group, indanyl group and binaphtyl group. The aryl group may be substituted by 1 to 3 substituants chosen independently ones of the others among an hydroxyl group, a linear or branched alkyl group containing from 1 to 4 carbon atoms as methyl, ethyl, propyl or preferably tert-butyle, a nitro group, a (C₁-C₄)alkoxy group and an halogen atom, as chore, bromine or iodine,

an <<arylalkyl group >> means preferably an aryl group appended to an alkyl group containing from 1 to 4 carbon atoms,

an <<alkoxycarbonyl group >> means preferably an alkoxy group containing from 1 to 4 carbon atoms appended to a carbonyl group, as methoxycarbonyl,

an <<heteroaryl group >> means preferably an aryl group containing from 1 to 3 heteroatoms, as nitrogen, sulphur or oxygen, including pyridyl, pyrazinyl, pyridazinyl, quinolyl, isoquinolyl, etc,

an <<heterocycle >> or <<heterocyclic group >> means preferably a 5- or 6-membered ring containing from 1 to 3 heteroatoms as sulphur, nitrogen, or oxygen. This definition also contains bicyclic rings where a heterocyclic group as previously defined is fused with a phenyl group, a cyclohexan group or any other heterocycle. Among heterocyclic groups imidazolyl, indolyl, isoxazolyl, furyl, pyrazolyl, thienyl, etc, may be cited,

an <<heteroarylalkyl group >> means preferably an heteroaryl group appended to an alkyl group containing from 1 to 4 carbon atoms, preferably methyl,

an <<heterocyclalkyl group >> means preferably an heterocyclic group appended to an alkyl group containing from 1 to 4 carbon atoms, preferably methyl, as 4-imidazolylmethyl.

More particularly, the ligand of formula (II) may be derived from:

an amino-alcohol of formula (III) 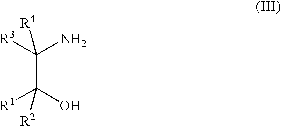
wherein R₁, R₂, R₃ and R₄ are as previously defined. Among amino-alcools of formulae (III) L-(S-(+)-) or D-valinol (R-(−)-2-amino-3-methyl-1-butanol), R-tert-leucinol (R-(−)-2-amino-5 3,3-dimethyl-1-butanol), S-tert-leucinol (S-(+)-2-amino-3,3-dimethyl-1-butanol), and (1S,2R)-(−)- or (1R,2S)-(+)-1-amino-2-indanol, may be cited,

an amino-ether of formula (IV) 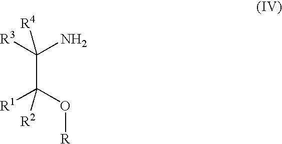
wherein R, R₁, R₂, R₃ and R₄ are as previously defined.

an amino acid of formula (V) 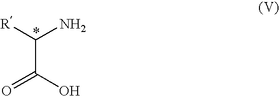
wherein R′ takes the definition of R₃ or R₄ as previously given. Among the amino acids of formulae (V) L-valin or D-valin, L-phenylalanin or D-phenylalanin, L-methionin or D-methionin, L-histidin or D-histidin and L-lysin or D-lysin may be cited.

an amino-ester of formula (VI) 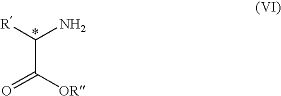
wherein R′ takes the definition of R₃ or R₄ as previously given and R″ takes the definition of R.

Preferably, in order to obtain particularly advantageous ligands, i.e. Schiff bases, these amino-alcohol, amino-ether, amino acids and amino-esters respectively of formulae (III), (IV), (V) and (VI) are reacted with an aldehyde of salicylic acid-of formula (VII) 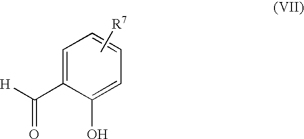
wherein R₇ represents from 1 to 2 substituents chosen independently ones of the others among an hydroxyl group, a linear or branched alkyl group containing from 1 to 4 carbon atoms such as methyl, ethyl, propyl or preferably tert-butyl, a nitro group, a (C₁-C₄)alkoxy group and an halogen atom, such as chlorine, bromine or iodine.

In the framework of the present invention, ligands of formula (II) are particularly preferred, said ligands are derived from an amino-alcool of formula (III), for which R₅ and R₆ represents together with the nitrogen atom a double bond —N═CHAr, wherein Ar is an aryl group containing from 1 to 3 substituents and at least an hydroxyl group, Ar being preferably a phenyl group,
R₁ and R₃, or R₂ and R₄, represent a hydrogen atom, whereas R₂ and R₄, or R₁ and R₃, respectively, are, independently ones of the others, linear or branched alkyl groups containing from 1 to 6 carbon atoms, preferably a tert-butyl group or form together a carbon cycle of 5 or 6 carbon atoms or a bicyclic ring system of 9 or 10 carbon atoms, wherein one of the cycles may be aromatic, preferably indanyl.

According to the present invention, a ligand may be advantageously chosen according to the catalyst used, and for example in the case of tungsten, a ligand may be used according to the seeked enantiomer, said ligand:

belonging to the family of quinine alcaloids as quinine, quinidine, dihydroquinidine (DHQD) or dihydroquinine (DHQ),

being derived from quinine alcaloids as hydroquinine 2,5-diphenyl-4,6-pyridinediyl diether (DHQ)₂-PYR or hydro-quinidine 2,5-diphenyl-4, 6-pyridinediyl diether (DHQD)₂-PYR.

In the case of a vanadium-based catalyst, a ligand represented by formula (II) above is preferably used, containing a substituant on the nitrogen atom, and for example a Schiff base derived from a substituted aldehyde of salicylic acid and from a chiral amino-alcool.

Generally, one uses preferably, in the case of a vanadium-based catalyst taken as vanadium acetylacetonate, a ligand derived form an amino-alcool or an amino-ether respectively of formulae (III) or (IV) as defined above. To the contrary, in the case of a vanadium-based catalyst taken as vanadium sulphate, a ligand derived from an amino acid or an amino ester respectively of formulae (V) or (VI) as defined above is preferably used.

Thus in the case of a vanadium-based catalyst, preferably taken as vanadium acetylacetonate, ligands 2,4-di-tert-butyl-6-[1-R-hydroxymethyl-2-methyl-propylimino)-methyl]-phenol and its isomer 2,4-di-tert-butyl-6-[1-S-hydroxymethyl-2-methyl-propylimino)-methyl]-phenol which allow to selectively orientate the reaction to the seeked enantiomer, are particularly preferred. Thus the use of 2,4-di-tert-butyl-6-[1-R-hydroxymethyl-2-methyl-propylimino)-methyl]-phenol allows to selectively orientate the oxidation reaction of the 5-methoxy-2-[[4-methoxy-3,5-dimethyl-2-pyridyl)methyl]thio]imi-dazo[4,5-b]pyridine to obtain the S-tenatoprazole, as indicated below.

In the same way, always in the case of a vanadium-based catalyst, preferably taken as vanadium acetylacetonate, ligand (1R,2S)-1-[2-hydroxy-3,5-di-tert-butyl-benzylidene)-amino]-indan-2-ol, derived from amino-indanol as amino-alcool, is particularly preferred.

Thus, the use of said ligand allows to selectively orientate the oxidation reaction of 5-methoxy-2-[[4-methoxy-3,5-dimethyl-2-pyridyl)methyl]thio]imidazo[4,5-b]pyridine, to selectively obtain the S-tenatoprazole, as indicated below.

Under the operating conditions, the ligand is preferably tridentate and forms with the metal catalyst an asymmetric complex where the metal is oxidized by the oxidant.

According to a characteristic feature of the present invention, the reaction may be carried out in a solvent, preferably in a mixture of solvents, in a neutral or weakly basic medium, by selecting a sulphide specific solvent and a ligand specific solvent, selected from the group consisting of methanol, tetrahydrofuran, methylene chloride, acetonitrile, toluene, acetone, chloroform, DMF (dimethylformamide) or NMP (N-methylpyrrolidinone), alone or in admixture. The base possibly used may be a tertiary amine such as pyridine, di-isopropylethylamine or triethylamine.

According to an alternative, the method may be implemented without the addition of a base, but it is preferable to avoid working in an acid medium as this could cause a degradation of the final product.

It is more particularly advantageous, according to the invention, to use the vanadium-based catalyst and the ligand in acetonitrile solution, whilst the sulphide is dissolved in a chlorinated solvent such as methylene chloride, and then the two solutions are mixed, and the oxidation is carried out.

The oxidation reaction is easily conducted at low temperatures or at room temperature. It might be advantageous to induce it at a temperature between 0 and 10° C. and preferably of about 4 to 5° C. in order to promote the enantioselectivity.

The method of the invention is particularly advantageous in as much as the oxidant and the catalyst are both widely commercially available, cheap and easy to process. Moreover, the catalyst can be used efficiently and in very small quantities. The yield of enantiomers obtained is excellent, and, moreover, the catalyst and the ligand can usually be recycled under good conditions without any loss of the enantiomeric excess.

The method of the present invention is particularly advantageous in the preparation of the enantiomers of tenatoprazole which can be represented by the following general formula:

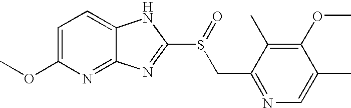

Thus, according to the method of the invention, a very advantageous enantioselective oxidation of 5-methoxy-2-[[(4-methoxy-3,5-dimethyl-2-pyridyl)methyl]thio]imidazo[4,5-b]pyridine by hydrogen peroxide in the presence of tungsten trioxide and of (DHQD)₂-PYR can be performed in order to obtain (−)-5-methoxy-2-[[(4-methoxy-3,5-dimethyl-2-pyridyl)methyl]sulfinyl]imidazo[4,5-b]pyridine.

More particularly, it has been noted that the oxidation of the above sulphide allows for the (−) enantiomer, having the S-configuration, to be obtained with excellent conditions of purity and yield when a vanadium-based catalyst is used in association with a ligand consisting of 2,4-di-tert-butyl-6-[1-R-hydroxy-methyl-2-methyl-propylimino)-methyl]-phenol or (1R,2S)-1-[2-hydroxy-3,5-di-tert-butyl-benzylidene)-amino]-indan-2-ol in acetonitrile solution, whilst the sulphide is in methylene chloride solution, or in acetone or in NMP respectively.

Conversely, the (+) isomer, having the R-configuration, can also be obtained with excellent conditions of selectivity and yield by using 2,4-di-tert-butyl-6-[1-S-hydroxy-methyl-2-methyl-propylimino)-methyl]-phenol or (1S,2R)-1-[2-hydroxy-3,5-di-tert-butyl-benzylidene)-amino]-indan-2-ol as a ligand.

The (−) and (+) enantiomers of tenatoprazole may be used under the form of salts, and particularly of alkaline metal salt or earth-alkaline metal salt, and for example under the form of a sodium, potassium, lithium, magnesium or calcium salts. These salts can be obtained from the (−) or (+) enantiomer of tenatoprazole which has previously been isolated by salification according to the standard method of the technique, for example by the action of basic mineral reagents comprising alkaline or earth-alkaline counter-ions.

Of course, the (−) and (+) enantiomers can be obtained in a pure optical form simply from the racemic mixture, using any appropriate method of separation, by preparative column chromatography, for example chiral or HPLC chromatography. The enantiomers thus obtained can be used for controls. “Pure optical form” means that the (−) enantiomer is substantially free from the (+) enantiomer, or only contains traces of it and vice versa. If necessary, a salification by a base is then performed in an appropriate solvent, in order to form a salt, and particularly an alkaline or earth-alkaline metal salt.

The principle of the chiral chromatography method is well known and is based on the difference in affinity existing between the (+) and (−) enantiomers and the chiral selector of the stationary phase. This method enables the separation of the enantiomers with a satisfactory yield.

The (−) enantiomer of tenatoprazole corresponds to (−)-5-methoxy-2-[[(4-methoxy-3,5-dimethyl-2-pyridyl)methyl]sulfinyl]imidazo[4,5-b]pyridine, or (−)-tenatoprazole. This form can be determined by optical rotation measurements using standard techniques. Thus, the optical rotation angle of the (−)-tenatoprazole is levo-rotatory in dimethylformamide an in acetonitrile, and its melting point is 130° (decomposition).

In the case of chiral separation of tenatoprazole, the racemic mixture used as the starting material can be obtained using known methods, for example according to the method described in Patent No. EP 254.588. Thus it can be prepared using an oxidizing agent, such as perbenzoic acid, to treat the corresponding sulphide arising from the condensation of a thiol and a pyridine, preferably in the presence of a base such as potassium hydroxide in an appropriate solvent, for example, ethanol, under heating.

In the treatment of the disorders mentioned below, the (−) and (+) enantiomers of tenatoprazole can be administered in standard forms adapted to the chosen administration route, for example per oral or parenteral route, preferably per oral or intravenous route.

For example, tablet or capsule formulations containing either one of the (−) and (+) enantiomers of tenatoprazole as an active substance, or else oral solutions or emulsions or 35 solutions for parenteral administration containing a tenatoprazole salt with a pharmaceutically acceptable standard substrate, can be used. The enantiomer salt of tenatoprazole can be chosen among the sodium, potassium, lithium, magnesium or calcium salts for example.

The (−) and (+) enantiomers of tenatoprazole obtained using the method of the present invention can be used in the manufacturing of drugs for the treatment of digestive disorders, and in particular of those where the gastric acid inhibition must be strong and prolonged, in the treatment of the symptoms and lesions of gastro-oesophageal reflux, digestive bleeding resistant to the other proton pump inhibitors.

The dosage regimen is determined by the physician according to the patient's state and the severity of the condition. It is generally between 10 and 120 mg, preferably between 20 and 80 mg, of (−) or (+) enantiomer of tenatoprazole per day.

Examples of the preparation of enantiomers are described below in order to illustrate the present invention without limiting its applications.

### EXAMPLE 1

### Preparation of (S)-(−)-tenatoprazole

10 g of WO₃, 73 g of (DHQD)₂-PYR, 3.5 L of THF and 330 g of 5-methoxy-2-[[4-methoxy-3,5-dimethyl-2-pyridyl)methyl]thio]imidaz[4,5-b]pyridine maintained under agitation at a temperature comprised between 4 and 5° C., are placed in a 5 L flask, and 120 mL of hydrogen peroxide at 30% are added thereto. The reaction medium is maintained under agitation for 48 hours. The catalyst is then filtered and the filtrate is diluted into 10 L of methylene chloride at room temperature.

The organic phase is washed with water, then dried and concentrated under reduced pressure. 242 g of the desired enantiomer are obtained, with an enantiomeric excess above 90% (70% yield).

A recrystallization is performed in the methanol/water or DMF/ethyl acetate mixture and the enantiomer is obtained with an enantiomeric excess superior to 99%. The enantiomeric excess is determined by high pressure liquid chromatography with a CHIRALPAK AS-H 20 μm (250×4,6 mm) column at 25° C., the eluent is acetonitrile (1 mL/min) and the detection is performed by U.V. spectroscopy at 305 nm. The retention time of the-(S)-(−) isomer equals 7.7 min, and that of the (R)-(+) isomer equals 5.2 min.
T_{F}: 129-130° C.
[α]²⁰D: −186.6 (c 0.1, DMF

Elementary analysis:

Elements

| | C | H | N | S |
|---|---|---|---|---|
| theory | 55.48 | 5.24 | 16.17 | 9.26 |
| observation | 55.66 | 5.22 | 16.16 | 9.37 |

UV Spectrum (methanol-water): λₘₐₓ: 272 nm (ε=6180), 315 nm (Ε=24877).

Infra-red (KBr): 3006, 1581, 1436, 1364, 1262, 1026, 1040 and 823 cm⁻¹.

RMN ¹H (DMSO d₆, reference:TMS) δ (ppm): 2.20 (s, 6H), 3.70 (s, 3H), 3.91 (s, 3H), 4.69-4.85 (m, 2H), 6.80 (d, J 8.5 Hz, 1H), 7.99 (d, J 8.5 Hz, 1 H), 8.16 (s, H), 13.92 (s, 1H)

RMN ¹³C (DMSO d₆, reference:TMS) δ (ppm): 13.2; 15.0; 56.6; 60.8; 62.6; 107.2; 129.5; 130.4; 131.9; 135.1; 150.5; 151.4; 156.9; 160.7; 163.0; 166.6.

### EXAMPLE 2

### Preparation of (R)-(+)-tenatoprazole

Using the same conditions as set out in Example 1, but replacing (DHQD)₂-PYR by (DHQ)₂-PYR, 120 mL of hydrogen peroxide are caused to react with the same quantity of 5-methoxy-2-[[(4-methoxy-3,5-dimethyl-2-pyridyl)methyl]thio]-imidazo[4,5-b]pyridine as set out in Example 1 and using the same catalyst.

The desired (+) enantiomer is thus obtained with an enantiomeric excess above 99%, after recrystallisation in a DMF/ethyl acetate mixture.

The rotatory power measured with a polarimeter in dimethyl formamide is [D]²⁰_{D}=+186°.

The physical and spectroscopic constants of (R)-(+)-tenatoprazole are identical to those of (S)-(−)-tenatoprazole, except for the specific rotatory power: [α]²⁰_{D}: +185.9 (c 0.1, DMF).

### EXAMPLE 3

### Preparation of (S)-(−)-omeprazole (esomeprazole)

Using the operating conditions of Example 1, and using 5-methoxy-2-[[(4-methoxy-3,5-dimethyl-2-pyridyl)methyl]thio]-1H-benzimidazole instead of 5-methoxy-2-[[(4-methoxy-3,5-dimethyl-2-pyridyl)methyl]thio]imidazo[4,5-b]pyridine, the desired product (esomeprazole) is obtained with an enantiomeric excess near 90% (72% yield).

The product obtained complies with the analytical data available in the literature.

### EXAMPLE 4

### Preparation of (S)-(−)-tenatoprazole

3 L of methylene chloride, and then 360 g of 5-methoxy-2-[[4-methoxy-3,5-dimethyl-2-pyridyl)methyl]thio]imidazo[4,5-b]pyridine are introduced in a 5 L flask. The mixture is left under stirring at room temperature for 30 minutes.

700 ml of acetonitrile, 5.22 g of 2,4-di-tert-butyl-6-[1-S-hydroxymethyl-2-methyl-propylimino)-methyl]-phenol, and then 2.90 g of vanadyl acetylacetonate are dropped one after the other in a 2 L flask. The mixture is kept under stirring at room temperature. After 30 minutes under stirring, this mixture is added to the preceding one.

135 ml of hydrogen peroxide at 30% are added to this mixture under stirring for 20 h at room temperature. After separation of the aqueous phase, the organic phase is washed twice with water, then dried and concentrated under reduced pressure. 283 g of the desired enantiomer are obtained, with an enantiomeric excess superior to 80% (75% yield). Two successive recrystallizations are performed in a methanol-/water or DMF/ethyl acetate mixture and the enantiomer is obtained with an enantiomeric excess higher than 99%.
T:127.5° C.
[α]²⁰_{D}: −182 (c 0.1, DMF)

### EXAMPLE 5

### Preparation of (R)-(+)-tenatoprazole

The instructions from Example 4 are followed but 2,4-di-tert-butyl-6-[1-R-hydroxymethyl-2-methyl-propylimino)-methyl]-phenol is replaced by 2,4-di-tert-butyl-6-[1-S-hydroxymethyl-2-methyl-propylimino)-methyl]-phenol.

The desired enantiomer is thus obtained.
[α]²⁰_{D}: +185.9 (c 0.1, DMF).

### EXAMPLE 6

### Preparation of (S)-(−)-tenatoprazole

1,2 L of NMP, and then 240 g of 5-methoxy-2-[[4-methoxy-3,5-dimethyl]-2-pyridyl)methyl]thio]imidazo[4,5-b]pyridine are introduced in a 5 L flask. The mixture is left under stirring at room temperature for 1 h30.

18 mL de NMP, 2,9 g of (1R,2S)-1-[2-hydroxy-3,5-di-tert-butyl-benzylidene)-amino]-indan-2-ol, and then 1,9 g of vanadium acetylacetonate are introduced in this order in a 50 mL round bottom flask. The mixture is stirred at room temperature. After 1 h30 of stirring, the solution is added into the reaction mixture.

Under stirring, 95 mL of hydrogen peroxide at 30% are added to this mixture for 20 hours at room temperature. The reaction mixture is precipitated by adding 500 mL of water.

The precipitate is recovered by filtration, then it is taken in 5 L of chloroform. The organic phase is washed twice with water, then dried and concentrated under reduced pressure. 126 g of the desired enantiomer are obtained with an enantiomeric excess superior to 30% (yield 50%). Several crystallizations in a DMF/ethyl acetate mixture are carried out and the enantiomer is obtained with an enantiomeric excess superior to 99%.

### EXAMPLE 7

### Preparation of (S)-(−)-tenatoprazole

3.7 L of acetone and then 30 g of 5-methoxy-2-[[4-methoxy-3,5-dimethyl]-2-pyridyl)methyl]thio]imidazo[4,5-b]-pyridine are introduced into a 10 L flask. The mixture is left under stirring for 30 minutes at room temperature.

30 mL of acetonitrile, 1.66 g of (1R,2S)-1-[2-hydroxy-3,5-di-tert-butyl-benzylidene)-amino]-indan-2-ol, and then 1.19 g of vanadium acetylacetonate are introduced in a 100 mL round bottom flask. The mixture is stirred at room temperature. After stirring for 1 h30, this suspension is added into the reaction mixture.

Under stirring, 10 g of urea-H₂O₂ dissolved in 7 mL of water and 50 mL of acetone are added for 6 hours to this mixture. Then, the mixture is left at room temperature for 12 hours. A sodium metabisulfite solution is added, then a 20% ammonia solution and acetone is concentrated. After washing with 100 mL of chloroform, the aqueous phase is collected and then neutralized with acetic acid. An extraction with 200 mL of chloroform is carried out twice. After separation of the aqueous phase, the organic phase is dried and concentrated under reduced pressure. 19 g of the desired enantiomer are obtained with an enantiomeric excess superior to 50% (yield 60%). Several crystallizations are carried out in a DMF/ethyl acetate mixture, and the enantiomer is obtained with an enantiomeric excess superior to 99%.

### EXAMPLE 8

### Preparation of (S)-(−)-tenatoprazole

4 L of acetone and then 30 g of 5-methoxy-2-[[4-methoxy-3,5-dimethyl]-2-pyridyl)methyl]thio]imidazo[4,5-b]pyridine are introduced into a 10 L flask. The mixture is left under stirring for 30 minutes at room temperature.

25 mL of acetonitrile, 1,66 g of (1R,2S)-1-[2-hydroxy-3,5-di-tert-butyl-benzylidene)-amino]-indan-2-ol, and then 1.19 g of vanadium acetylacetonate are introduced in this order in a 100 mL round bottom flask. The mixture is stirred at room temperature. After stirring for 1 h30, this suspension is added into the reaction mixture.

Under stirring, 30 g of sodium sulfate, 10 g of urea-H₂O₂ dissolved in 7 mL of water and 50 mL of acetone are added for 6 hours to this mixture. Then, the mixture is left under stirring at room temperature for 12 hours. A sodium metabisulfite solution is added, then a 20% ammonia solution and acetone is concentrated. After washing with 100 mL of chloroform, the aqueous phase is collected and then neutralized with acetic acid. An extraction with 200 mL of chloroform is carried out twice. After separation of the aqueous phase, the organic phase is dried and concentrated under reduced pressure. 20.1 g of the desired enantiomer are obtained with an enantiomeric excess superior to 65% (yield 64%). Several crystallizations are carried out in a DMF/ethyl acetate mixture, and the enantiomer is obtained with an enantiomeric excess superior to 99%.

1. A method for the enantioselective preparation of sulfoxides derivatives or basic salts thereof comprising:
(a) enantioselective oxidation of a sulphide of the following general formula (I)
A-CH₂—S—B (I)
wherein
A is a diversely substituted pyridyl nucleus and
B a heterocyclic residue comprising a benzimidazole or a imidazo-pyridyl nucleus, using an oxidizing agent in the presence of a tungsten- or vanadium-based catalyst and of a chiral ligand;
(b) optionally salification by a base, in order to obtain the sulfoxide A-CH₂—SO—B (Ia).

2. A method according to claim 1, wherein, in general formula (I), A is a pyridyl group or a pyridyl group bearing one or more substituents selected from the linear or branched alkyl groups of 1 to 6 carbon atoms, linear or branched alkoxy groups of 1 to 6 carbon atoms, methyl or ethyl groups substituted by one or several halogen atoms, amino, alkylamino or dialkylamino groups where the alkyl moiety, whether linear or branched, comprises 1 to 5 carbon atoms; B represents a heterocycle selected from the benzimidazole or imidazo-[4,5-b]-pyridyl groups, optionally substituted by one or several linear or branched alkyl groups of 1 to 6 carbon atoms, linear or branched alkoxy groups of 1 to 6 carbon atoms.

3. A method according to claim 2, wherein the A and B groups are substituted on one or several carbon atoms by a methyl, ethyl, methoxy or trihalogenomethyl group.

4. A method according to claim 3, wherein A is a 2-pyridyl group substituted by one or several methyl, ethyl, methoxy or trifluoromethyl groups.

5. A method according to claim 3 wherein A is a 4-methoxy-3,5-dimethyl-2-pyridyl group and B is a 5-methoxy-1H-benzimidazolyl or 5-methoxy-imidazo-[4,5-b]-pyridyl group.

6. A method according to claim 1, wherein the obtained enantiomer is salified by reaction with basic mineral reagents comprising alcaline or earth-alcaline counter ions.

7. A method according to claim 6, wherein the salt is a sodium, potassium, lithium, magnesium or calcium salt.

8. A method according to claim 1, wherein the oxidizing agent is a peroxide or a hydroperoxide.

9. A method according to claim 8, wherein the oxidizing agent is hydrogen peroxide, urea-H₂O₂ (UHP) or cumene or tertiobutyl hydroperoxide.

10. A method according to claim 1, wherein the catalyst is a (V) oxo-vanadium complex or a derivative of tungsten.

11. A method according to claim 10, wherein the complex or the derivative is prepared from tungsten trioxide, vanadium acetylacetonate, or vanadium sulphate.

12. A method according to claim 1, wherein the catalyst is vanadium based and the ligand is tridentate.

13. A method according to claim 1, wherein the ligand is represented by the following general formula (II):
RO—CR₁R₂—CR₃R₄—NR₅R₆
where
R is a hydrogen atom or a linear or branched alkyl group of 1 to 6 carbon atoms or an aryl or heteroaryl group;
R₁ to R₄, which can be the same or different, represent a linear or branched alkyl group of 1 to 6 carbon atoms, possibly optionally comprising a heteroatom such as selected from sulphur, nitrogen and oxygen and/or and optionally substituted by an amino group; an aryl group; an alkylaryl group; an alkoxycarbonyl group; a heteroaryl group or a heterocyle; a heteroarylalkyl or a heterocyclalkyl group,
with the proviso that R₁ should not be identical with R₂, and/or R₃ should not be identical with R₄, so that the ligand comprises one, or two asymmetry centers;
R₁ and R₂ together can represent a carbonyl group C═O;
R₁ and R₃, or R₂ and R₄ together, can form a carbon ring having 5 or 6 carbon atoms or a bicyclic system with 9 or 10 carbon atoms where one of the cycles can be aromatic;
R₄ and R₅, which can be the same or different, can form a 5- or 6-membered heterocycle with the nitrogen atom;
R₅ and R₆, which can be the same or different, represent a linear or branched alkyl group of 1 to 6 carbon atoms or a 5 or 6-membered carbon ring, or form a heterocycle with the nitrogen atom to which they are bound, or
R₅ and R₆ represent, together with the nitrogen, a —N═CHAr double bond where Ar is a aryl residue, possibly optionally substituted by 1 to 3 groups, and preferably bearing a hydroxyl group.

14. A method according to claim 13, wherein Ar is a 2′-hydroxyphenyl group optionally substituted on the aryl group.

15. A method according to claim 13, wherein:
R₁ and R₃ or R₂ and R₄ represent an hydrogen atom, whereas R₂ and R_{b 4} or R₁ and R₃, respectively, are linear or branched alkyl groups of 1 to 6 carbon atoms, a aryl group or form together a carbon ring having 5 or 6 carbon atoms or a bicyclic system with 9 or 10 carbon atoms where one of the cycles can be aromatic.

16. A method according to claim 13, wherein the aryl group is selected from a phenyl group, a naphtyl group, a tetrahydronaphtyl group, an indanyl group and a binaphtyl group, where the aryl group can be substituted by 1 to 3 substituents selected from a hydroxyl group, a linear or branched alkyl group comprising 1 to 4 carbon atoms, a nitro group, a (C₁-C₄)alkoxy group and a halogen atom.

17. A method according to claim 13, wherein the ligand of formula (II) is alternatively derived from:
an amino alcohol of formula (III) 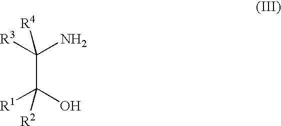
wherein R₁, R₂, R₃ and R4 are as defined claim 13,
an amino-ether of formula (IV) 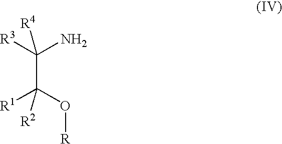
wherein R, R₁, R₂, R₃ and R₄ are as defined in claim 13,
an amino acid of formula (V) 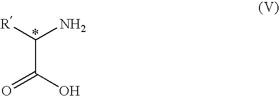
wherein R′ takes the definition of R₃ or R4 according to claim 13 or,
an amino-ester of formula (VI) 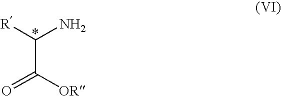
wherein R′ takes the definition of R₃ or R₄ according to claim 13 and R″ takes the definition of R according to claim 13.

18. A method according to claim 17, wherein the amino-alcohol of formulae (III) is selected from L- or D-valinol, R-tert-leucinol, S-tert-leucinol and (1S,2R)-(−)- or (1R,2S)-(+)-1-amino-2-indanol and in that the amino acid of formulae (V) is selected from L-valine or D-valine, L-phenylalanine or D-phenylalanine, L-methionine or D-methionine, L-histidine or D-histidine, L-lysine or D-lysine.

19. A method according to claim 17, wherein the ligand of formula (II) is obtained by reacting an amino-alcohol, an amino-ether, an amino acid or an amino-ester of formulae (III), (IV), (V) and (VI), respectively, as defined in claim 17 with an aldehyde of salicylic acid, of formula (VII) 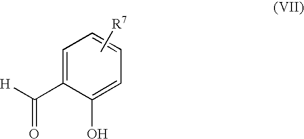
wherein R₇ represents 1 to 2 substituents independently selected from an hydroxyl group, a linear or branched alkyl group containing from 1 to 4 carbon atoms, a nitro group, a (C₁-C₄)alkoxy group and a halogen atom.

20. A method according to claim 17, wherein a catalyst prepared from vanadium acetylacetonate and a ligand derived from an amino-alcohol or an amino-ether respectively of formulae (III) or (IV) as defined in claim 17, are used.

21. A method according to claim 20, wherein the ligand of formula (II) is derived from an amino-alcohol of formula (III) as defined in claim 17, for which
R₅ and R₆ represent together with the nitrogen atom a double bind —N═CHAr, wherein Ar is an aryl group containing from 1 to 3 substituents with at least one of which being an hydroxyl group,
R₁ and R₃, or R₂ and R₄, represent an hydrogen atom, whereas R₂ and R₄, or R₁ and R₃, respectively, are, independently selected from, linear or branched alkyl groups of 1 to 6 carbon atoms, preferably a tert-butyl group or form together a carbon cycle of 5 or 6 carbon atoms or a bicyclic ring system of 9 or 10 carbon atoms wherein one of the cycles may be aromatic.

22. A method according to claim 17, wherein a catalyst prepared from vanadium sulphate and a ligand derived from an amino acid or an amino-ester respectively of formulae (V) or (VI), as defined in claim 17 are used.

23. A method according to claim 1, wherein the ligand is 2,4-di-tert-butyl-6-[1-R-hydroxymethyl-2-methyl-propylimino)-methyl]-phenol, le 2,4-di-tert-butyl-6- [1-S-hydroxymethyl-2-methyl-propylimino)-methyl]-phenol, le (1R, 2S)-1-[2-hydroxy-3,5-di-tert-butyl-benzylidene)-amino]-indan-2-ol or (1S, 2R)- 1-[2-hydroxy-3,5-di-tert-butyl-benzylidene)-amino]-indan-2-ol.

24. A method according to claim 23, wherein the ligand is in an acetonitrile solution.

25. A method according to claim 23 wherein an enantioselective oxidation of 5-methoxy-2-[[(4-methoxy-3,5-dimethyl-2-pyridyl)methyl]thio]imidazo [4,5-b]pyridine is carried out to obtain (−)-5-methoxy-2-[[(4-methoxy-3,5-dimethyl-2-pyridyl)methyl] sulfinyl]imidazo [4,5-b]pyridine by using a vanadium-based catalyst associated with a ligand consisting of 2,4-di-tert-butyl-6-[1-R-hydroxymethyl-2-methyl-propylimino)-methyl]-phenol or (1R, 2S)- 1-[2-hydroxy-3,5-di-tert-butyl-benzylidene)-amino]-indan-2-ol in an acetonitrile solution, whilst the sulphide is in a methylene chloride or acetone or N-methylpyrrolidinone solution, respectively.

26. A method according to claim 10 wherein the catalyst is a tungsten derivative and the ligand is hydroquinine 2,5-diphenyl-4,6-pyridinyl diether (DHQ)₂-PYR or hydroquinidine 2,5-diphenyl-4,6-pyridinyl diether (DHQD)₂-PYR.

27. A method according to claim 26, wherein an eniantoselective oxidation of 5-methoxy-2-[[(4-methoxy-3,5-dimethyl-2-pyridyl)methyl]thio]imidazo [4,5-b]pyridine is carried out by hydrogen peroxide in the presence of tungsten trioxide and of (DHQD)₂-PYR in order to obtain the (−)-5-methoxy-2-[[(4-methoxy-3,5-dimethyl-2-pyridyl) methyl]sulfinyl]imidazo [4,5-b]pyridine.

28. A method according to claim 1 wherein the oxidation reaction is carried out in a solvent, in a neutral or weakly basic medium.

29. A method according to claim 28, wherein the solvent is a mixture of solvents comprising a sulphide specific solvent and a ligand specific solvent selected from methanol, tetrahydrofuran, dichloromethane, acetonitrile, toluene, acetone, chloroform, dimethylformamide and N-methylpyrrolidinone, alone or in admixture, and the base is a tertiary amine selected from pyridine, di-isopropylethylamine and triethylamine.

30. A method according to claim 13 wherein Ar is substituted by 1 to 3 hydroxyl groups.
